(19) United States
(12) Patent Application Publication
(10) Pub. No.: US 2006/0173381 A1
(43) Pub. Date: Aug. 3, 2006

(54) Catheter

(76) Inventor: Kai Eck, Aachen (DE)

Correspondence Address: PHILIPS INTELLECTUAL PROPERTY & STANDARDS, P.O. BOX 3001, BRIARCLIFF MANOR, NY, 10510, US

(21) Appl. No.: 10/536,062

(22) PCT Filed: Nov. 24, 2003
(86) PCT No.: PCT/IB03/05359

§ 371 Date: May 24, 2005

(30) Foreign Application Priority Data
Nov. 30, 2002 (DE) 10256007.2

(51) Int. Cl. A61M 25/00

(52) U.S. Cl. 600/585

(57) ABSTRACT

The invention relates to a catheter arrangement which comprises at least a first basic element and a second basic element, which second basic element is arranged such that it is slidably arranged in the first basic element over at least part of its length and has a sensor unit that is provided for determining a position and/or mutual position shift of the first basic element and the second basic element, to generate at least a sensor value that is assigned to a measurable property of the catheter arrangement. The repeated reaching of a once-defined position is allowed by such a catheter arrangement, so that after expanding a vessel constriction, a stent can be taken to the same position without having to take X-rays for verification of the position, which are burdensome to the patient.

The invention relates to a catheter arrangement comprising at least two basic elements.

Catheters, which are used in medical technology for diagnostic or surgical processes, have at least two basic elements: a catheter sleeve and an instrument catheter, which is moved within the catheter sleeve after placing it in the catheter sleeve, so that the instrument which the instrument catheter typically has on its tip is pushed into the desired place inside the patient and is then located is outside the catheter sleeve. A catheter, as is used, for example, in intercoronary arterial operation, mainly comprises three basic elements, namely a catheter sleeve, which has a relatively large diameter (about 2-3 mm), a guide wire with a relatively small diameter (about 0.25 mm) and an internal catheter, which can be moved inside the catheter sleeve over the guide wire. Catheter arrangements with two or three basic elements are also known in other applications, such as for minimum invasive interventions or in endoscopic examinations. The size ratios differ from those for intercoronary arterial applications, according to the application.

In an intercoronary arterial operation, the catheter sleeve is introduced in an artery in the groin or the shoulder of the patient and is pushed up to the heart as far as the ostium. The catheter sleeve cannot be pushed any further in this application due to its large diameter. The guide wire, typically with an elastic head, is moved further into the coronary arteries, till the guide wire tip has been moved ahead behind the arterial region to be treated (for example an arterial constriction). The positioning is done with the help of X-ray fluoroscopy sequences with a contrast medium, to make the coronary artery free and the anomaly to be treated (for example a constriction) appear in the fluoroscopy images., Besides the contrast medium injection also X-rays for positioning are made. Both are burdensome to the patient.

First an internal catheter is moved over the guide wire. It typically bears an instrument on its tip, about an inflatable balloon, by means of which the coronary artery constriction can be expanded. In a second step, the internal catheter is pulled out again and a second internal catheter is introduced, which has what is called a stent, which is a thin wire mesh, used for stabilizing the expanded portion of the arterial area. The stent must then come to be at the same location, at which also the artery was expanded. This is again done typically with the help of fluoroscopy recorded images. These are an additional burden to the patient and it would be desirable to reduce this burden.

It is therefore an object of the invention to improve the catheter management, so that the burden to the patient is reduced.

The object is achieved through a catheter arrangement, which comprises at least a first basic element and a second basic element, which is movably arranged over at least a part of its length within the first basic element, and which catheter arrangement has a sensor unit provided for generating at least a sensor value which is assigned to a measurable property of the catheter arrangement, for determining a position and/or a position shift of the first basic element and the second basic element to each other.

The advantage of the invention as claimed in claim 1 is that it renders possible the determination of the position or the mutual position shift of two basic elements, say, the position of the inner catheter relative to the guide wire or to the catheter sleeve. A position reached once can thus be easily reached again without needing the burdensome X-rays. This takes place by comparing two position shifts or positions. If the first instrument (for example the balloon) has been placed, then either the shift is measured or the position at the location of use of the instrument is measured when the instrument catheter is pulled out. If a second instrument catheter, with its instrument (for example the stent) is pushed in again, the same position shift can be made in another direction or the shifting is done till the same position is measured again. The sensor unit used for measuring then measures a measurable property. The measurable property can be regular markings, which can be measured electromagnetically, mechanically or optically, magnetically recorded information or only the property of the guide wire, having a certain resistance in a power circuit from one end up to the position of the sensor, which resistance can be assigned to a position. Depending upon the type of the measurable property, it allows to determine a position by means of a sensor value or to determine a position shift by means of two or more sensor values (this includes a continuous reading of the sensor values).

Claim 2 shows a particularly advantageous embodiment. Using a sensor, which is located on one of the basic elements, a measurable property shown by another basic element can be measured and the sensor values can be converted into a position value or a position shift value.

A special embodiment of the measurable property is a structuring. A structuring can be a mechanical, electromagnetic or optical property.

Another advantageous embodiment of the invention is provided if the structure of the structured basic elements can be measured without touching them, because contact always entails wear and tear and mechanical resistance, which can be avoided by contactless measuring.

Another advantageous embodiment of the invention is provided if the structuring of the structured basic elements varies in the longitudinal direction, as described in claim 5. This, for example, indicates having a uniform structure, which allows a position shift determination by simple counting off of the measured rings.

The invention can have a particularly advantageous embodiment if there are two sensors, which measure the regular structuring. Because if the distance between the two sensors is smaller than the width of the structuring; the direction of movement can be determined and multiple pulling forward or backward in the positioning operation can be taken into account.

If the regular structurings are structurings of the electromagnetic properties, as described in claim 6, then it is mostly easy to realize the sensor, for example, as a simple contact or as a capacitance measuring sensor. If the electromagnetic property is the connectivity, the structuring can also be realized easily, say, by simple insulation.

In another advantageous embodiment of the invention the sensor unit has a sensor evaluation unit, which can convert the sensor values of the first sensor into position values or into position shift values.

A typical embodiment of the catheter arrangement has a basic element that is elongated and hollow, so that it is easy to achieve a shift capability for a second basic element in the first basic element.

The invention further relates to a method for determining the position and/or a position shift of a first basic element and a second basic element of a catheter arrangement, in which method the second basic element is arranged so that it can be moved over at least a portion of its length in the first basic element, in which a sensor unit generates at least a sensor value which is assigned to a measurable property of the catheter arrangement.

These and the other aspects of the invention are apparent from and will be elucidated with reference to the embodiments described hereinafter. In the drawings,

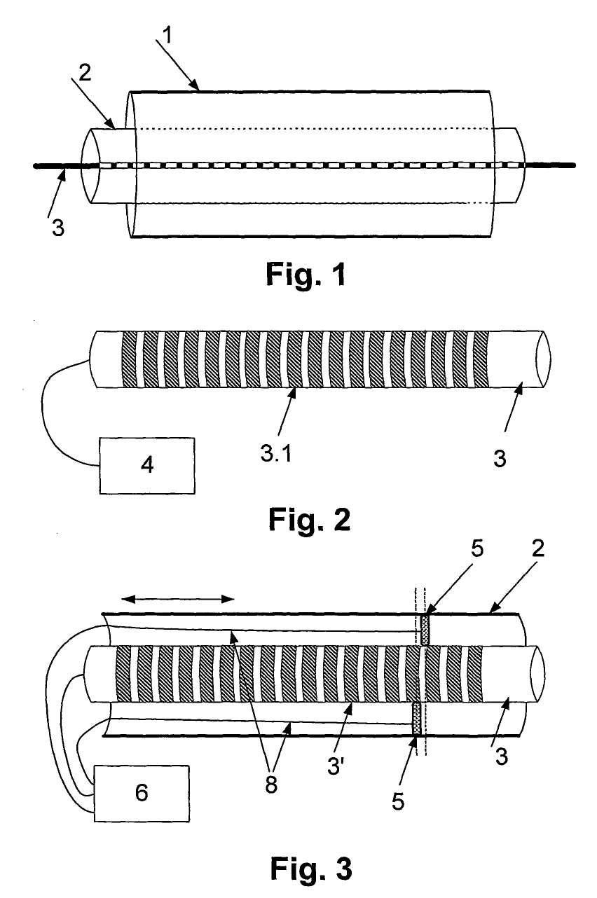

FIG. 1 schematically shows a catheter arrangement comprising the three basic elements that can be moved into each other.

FIG. 2 schematically shows a structuring of the guide wire, where the guide wire is also linked to a supply unit,

FIG. 3 shows the inner catheter, which can be shifted in longitudinal direction relative to the guide wire and has two sensors in contact with the guide wire which are connected to a sensor evaluation unit, and

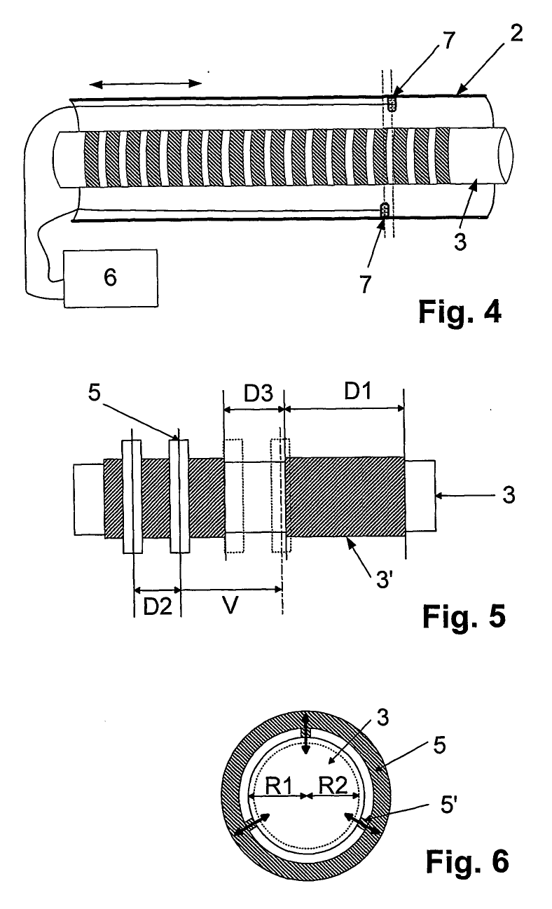

FIG. 4 shows an inner catheter and a structured guide wire, where the inner catheter has two sensors which measure the structuring of the guide wire without contact,

FIG. 5 shows a side detail of a structured guide wire, that has three structural elements and two ring electrodes that are arranged on the inner catheter, which is not drawn, and

FIG. 6 shows a cross-section through the structured guide wire and a ring electrode with three contact points.

A catheter arrangement comprising the basic elements, catheter sleeve, inner catheter and guide wire, is used for intercoronary arterial applications in the blood—filled arteries of a patient. It will be depicted below, how such a catheter arrangement can be arranged to make it possible to determine a position value or a position shift value, resulting in less burden on the patient, if a position has to be reached more than once. The catheter arrangement need not, however, be restricted to three basic elements, because the invented embodiment also functions with two or more than three basic elements.

FIG. 1 schematically shows the three basic elements of a catheter arrangement for intercoronary arterial applications. The catheter sleeve 1 is the outermost of the three basic elements. Within the catheter sleeve there is the inner catheter 2 which can be moved over a guide wire 3 (also called guide wire). The guide wire 3 is the innermost of the three basic elements. The three basic elements often have different lengths (e.g. the guide wire 3 is typically designed longer than the catheter sleeve 1). The three basic elements are arranged to be capable of moving essentially independently of each other.

FIG. 2 schematically shows a structuring of the guide wire 3. Structurings 3′ are provided on the guide wire 3 or incorporated with it. Another embodiment is the measurable property of the Ohmic resistance between a fixed contact point and the point defined by a shiftable contact. In the embodiment shown the guide wire 3 is connected to a supply unit 4, which makes it possible to maintain the guide wire 3 continuously at a voltage potential or to supply it with energy (e.g. in the form of current) or light.

FIG. 3 shows the structured guide wire 3 with the inner catheter 2 (cut away for clarity). On the inner catheter 2 in this embodiment, there are sensors 5 which measure the structuring 3′ of the guide wire 3 by contacting and thereby make it possible to determine the mutual position shift of inner catheter 2 and guide wire 3. If the sensor value, however, is a measure for the length between a fixed point and the location of the sensor (in the embodiment not shown here this would be the Ohmic resistance), then it will be possible to determine a position of only one single sensor value. FIG. 3 further shows a sensor evaluation unit 6, which records the sensor values of the sensors 5 and on the basis of these sensor values, and possible other fixed parameters, determines the position and/or position shift of the two basic elements with respect to each other. The sensors 5 are contacted by means of supply wires 8. In the sensor evaluation unit 6 also voltage sources, if required, and similar supply sources should be integrated in such a manner that no additional supply unit 4 is needed.

If the guide wire 3 is arranged such that it transmits light on the structures 3′, the structures can be measured by means of an optically sensitive sensor, such as a photo diode for example. For this purpose, the guide wire 3 itself can radiate in that it is made from a lucent material or a material that can be excited to luminescence. The guide wire may also be a light conductor, however, where light is coupled out at the structures 3′. If the guide wire 3 comprises an optically transparent material, to which a phosphorescent material has been added, the guide wire can be excited to luminescence by prior exposure to light. A structuring can be realized by optically opaque covers. If the inner catheter 2 provided with a photo diode is slid over the guide wire 3, then the output signal of the photo diode would increase each time it begins to travel over a lighting structure. The output signal will then drop again as the photo diode is slid over an optically opaque cover.

Another embodiment of the guide wire 3 is obtained if the guide wire consists of a material whose electrical conductivity is considerably higher than that of blood (this becomes necessary, because the catheter is at least partly filled with blood in intercoronary arterial applications). The guide wire can be made of metal or some other conductive material or mixture of materials, such as a conductive plastic or a plastic metal mixture. To realize a structuring of such a guide wire, the guide wire is given an insulating coating, which is removed or not deposited respectively at places for realizing the structures 3′. The inner catheter 2 has at least a (ring-shaped) electrode, which is contacted by means of a flexible supply wire 8. The supply wire 8 is incorporated in the inner catheter 2, which can be realized, for example, during the manufacturing process of the inner catheter 2 by extrusion, or it is just glued onto it. The supply wire 8 can be contacted at the other end of the inner catheter 2 (therefore typically outside the patient). It is linked to the sensor evaluation unit 6 in the embodiment depicted here. The electrode is designed in such a manner that it slides over the guide wire when the inner catheter is moved forward and backward and comes into contact with the non-insulated tips. This can be realized, for example, by means of resilient contacts or through brush contacts. The contacting need not take place on the entire periphery. To ensure a good contact every time, however, three contact points are advisable on a circular ring electrode enveloping the guide wire 3 (see FIG. 6). At these points, a smaller resistance is measured between guide wire and electrode than at the insulated points, if the guide wire and electrode are linked to a voltage source. The internal resistance values of the voltage source and the voltage must be selected appropriately so that only low currents are measured and there are no disturbing effects on the patient's body.

In a special embodiment of the invention, as shown by means of FIGS. 5 and 6, the structurings 3′ with a width D3 are arranged on the guide wire 3. These structurings 3′ are points without insulation of the conductive material from which the guide wire is manufactured. FIG. 5 shows a side view of the structured guide wire 3 with two ring electrodes 5. The inner catheter 2, on which the ring electrodes 5 are provided and the supply wires 8 of the electrodes are not shown here for the sake of simplicity. FIG. 6 shows a cross section through the guide wire with a ring electrode and the contact points 5′ arranged on it. In this version, the two ring electrodes 5 have three contact points 5′ each made of resiliently arranged warpings of electrode material, arranged on the periphery of the ring electrodes 5. The respective double arrows show an elasticity of the contact points 5′ in the radial direction, such that the contact points follow the changing radii of the guide wire at the insulated (radius R1) and the non-insulated points (shown by the dashed periphery of the guide wire 3 in FIG. 6; at these points the guide wire 3 has the smaller radius R2), without losing contact to the surface of the guide wire. The contact points 5′ are mutually offset by 120°, so that contact to the guide wire 3 is always ensured. Contact points 5′, which can adapt to a changing diameter, can also be made as brush contacts. The two ring electrodes 5 are arranged at a center-to-center distance D2 on the inner catheter 2, not shown, and contacted by means of supply wires. The length of the insulated points on the guide wire 3 is D1. D1 and D3 are selected in the version described here, so that they are always larger than the center-to-center distance D2 between the ring electrodes. The result of this is that there is always a position on an insulated or on a non-insulated structure where both electrodes 5 either have contact with the guide wire 3 or no contact.

By means of the two ring electrodes 5 positioned at center distance D2, the position shift of inner catheter 2 with respect to guide wire 3 can be determined. In the initial position drawn here, both ring electrodes 5 have no contact to one of the structurings 3′ and therefore a high resistance is measured on both ring electrodes. If the inner catheter 2 is moved on the guide wire 3 in the direction of the arrow V, then the electrode arranged forward seen in shift direction V first comes into contact with the non-insulated structuring and then the electrode arranged at the back seen in shift direction V comes into contact so that a low resistance to the guide wire 3 is measured in the dashed shifting position of the ring electrodes for both electrodes. If the move continues in the direction of shift V, then the electrode arranged in front loses contact first and then the electrode arranged at the back in the direction of shift loses contact. The sensor evaluation unit 6 then simply counts the structurings 3′ traveled past and a value corresponding to it (e.g. the actual shift distance which can be computed by means of the fixed given values of D3 and D1) can be displayed to the user of the catheter arrangement, for example, on a display on the sensor evaluation unit 6. The special arrangement of the ring electrodes and structurings in this version makes it possible for the sensor evaluation unit 6 to recognize whether the direction of shift is changed during the shift. If, for example, the direction of shift is changed when both electrodes have no contact, then the next contact is measured on the electrode being at the back in the former direction of shift, which can be recognized by the sensor evaluation unit 6. Similarly, the electrode being at the back in the former direction of shift loses contact first, if the direction of shift is changed, while both electrodes have contact. If the direction of shift is changed while only one electrode has contact, this also leads to a recognizable deviation from the behavior as has been described for a constant direction of shift. The precision of positioning achieved depends on the selected distances D1, D2 and D3. For intercoronary arterial applications, a position determination of about one millimeter is sufficient and D1 could be one millimeter, D3 half a millimeter and D2 one third millimeter. According to the requirements and technical boundary conditions, other values could also be selected. The determination of the position or position shift can become more accurate by assessment of the sensor value (both electrodes have contact, only one electrode has contact, both electrodes have no contact) than by just counting the structures passed. If D1 and D3 are known, then the shift distance during counting can be indicated in units of D1+D3. If the contact signals are evaluated, then on loss of contact for both electrodes, an intermediate value of about (D1+D3)/2, can be added to the shift path. The inaccuracy of this information depends on the distance values selected.

If it is required that the inner catheter 2 is to be moved without changing the direction of shift, then an embodiment with only one electrode 5 is of advantage, because then only the number of structured points 3′ needs to be counted to achieve the determination of a position shift. Such a version is simpler and more cost-effective to make from the point of view of manufacturing technology. If the direction of shift changes, this can be communicated to the sensor evaluation unit 6, for example, manually, by pushing a button. Subsequently, the change in the shift distance is counted in the other direction on the basis of the counted structurings 3′.

In another embodiment the ring electrodes 5 are embodied without the contacting points 5′. Without a direct or conductive contact between ring electrodes 5 and structurings 3′ the structurings passed can be measured capacitively. In this contactless embodiment, it is advantageous if the voltage supply is not DC voltage but provided by a high-frequency source. Furthermore, embodiments with inductive measurement can be made, where the ring electrodes 5 are replaced by coils, which have two supplies each. Accordingly, the structurings 3′ are to be executed as coils on the guide wire 3.

1. A catheter arrangement, which comprises at least a first basic element and a second basic element which is movably arranged over at least a part of its length within the first basic element, and which catheter arrangement has a sensor unit provided for generating at least a sensor value which is assigned to a measurable property of the catheter arrangement, for determining a position and/or a position shift of the first basic element and the second basic element to each other.

2. Catheter arrangement, as claimed in claim 1, wherein the sensor unit has at least a first sensor, which is arranged on one of the basic elements and in that another one of the basic elements has the property measurable by the first sensor.

3. Catheter arrangement as claimed in claim 2, wherein the measurable property is a structuring.

4. Catheter arrangement as claimed in claim 2, wherein the first sensor is provided for contactless measurement of the measurable property.

5. Catheter arrangement as claimed in claim 3, wherein the structuring is a regular structuring varying in the direction of the possible mutual shift of the first and second basic elements.

6. Catheter arrangement as claimed in claim 5, wherein at least a second sensor is arranged on the same basic element Was the first sensor sand both sensors are provided to measure the regular structuring.

7. Catheter arrangement as claimed in claim 6, wherein the regular structuring is a structuring of an electromagnetic property.

8. Catheter arrangement as claimed in claim 2, wherein the sensor unit has a sensor evaluation unit, which is coupled to the first sensor and which is provided for determining the position and/or position shift from the sensor value.

9. Catheter arrangement as claimed in claim 1, wherein the first basic element is embodied as elongated and hollow.

10. Method for determining a position and/or position shift of a first basic element and a second basic element of a catheter arrangement to each other, in which method the second basic element is arranged such that it can be moved over at least a portion of its length in the first basic element, in which a sensor unit generates at least a sensor value that is assigned to a measurable property of the catheter arrangement.
